(19) United States
(12) Patent Application Publication
(10) Pub. No.: US 2023/0089228 A1
(43) Pub. Date: Mar. 23, 2023

(54) Personal Care Compositions

(71) Applicant: Colgate-Palmolive Company, New York, NY (US)

(72) Inventors: Yanjun DENG, Guangzhou (CN); Yuyan ZENG, Guangzhou (CN); Dong Yao HUANG, Guangzhou (CN); Changlong CHEN, New Brunswick, NJ (US); Hongwei SHEN, Holmdel, NJ (US)

(21) Appl. No.: 17/941,484

(22) Filed: Sep. 9, 2022

(30) Foreign Application Priority Data
Sep. 9, 2021 (CN) 202111054705.8

(51) Int. Cl. A61K 8/41; A61Q 19/10; A61K 8/42; A61K 8/46

(52) U.S. Cl. CPC A61K 8/416; A61Q 19/10; A61K 8/42; A61K 8/463

(57) ABSTRACT

Disclosed are personal care compositions, e.g., natural liquid soap compositions, comprising an effective amount of a long chain alkyl (C₆-C₂₂) anionic surfactant (e.g., disodium 2-sulfolaurate), an effective amount of an amphoteric surfactant (e.g., cocamidopropyl betaine), an effective amount of one or more alkyl glucoside(s) (e.g., decyl glucoside and coco-glucoside); and water, as well as to methods of using these compositions.

### CROSS-REFERENCE TO RELATED APPLICATIONS

The instant application is a U.S. Non-Provisional Application which claims priority to Chinese Application No. 202111054705.8, filed on Sep. 9, 2021, the contents of which are herein incorporated by reference in its entirety.

### BACKGROUND

Liquid cleansing compositions, such as aqueous foaming compositions like hand soaps, body washes, and shower gels, should desirably have acceptable levels of foam production for consumer satisfaction. Foam production, for example, is a key attribute for consumers when choosing products. To achieve a desirable foam production, manufacturers incorporate sufficient quantities of foaming agents, such as surfactants, into the compositions.

However, in recent years, consumer demand for more natural products has increased—e.g., interest in the origin of raw materials and products with ingredients with a high degree of naturality. However, it is a challenge to produce formulations that have the physical characteristics that consumers expect, for example foam production with the right viscosity, while at the same time including products with a high degree of naturality. Moreover, liquid consumer products normally contain different species of surfactants. One example can be the mixture of sodium laureth sulfate (SLES) and cocamidopropyl betaine (CAPB), which constitutes the backbone in a variety of personal care products. The SLES/CAPB mixture is desirable given its foamability and viscosity characteristics. However, with the increasing concern regarding sulfate surfactants, and 1,4-dioxane impurity from ethoxylation in personal care products, sulfate-free/ethoxylation-free shower gel and shampoos are believed to be becoming more desirable to consumers in the personal care industry. Consequently, manufacturers are shifting to the use of natural/sustainable surfactant substitutes. One challenge with surfactant substitutes, however, is thickening these formulations without involving other thickening agents, e.g., polyacrylic acid (Carbomer), xanthan gum, polyethylene glycol, and cocoamide MEA.

Accordingly, there is a need for a personal care product that is both natural but still retains acceptable physical characteristics for consumer satisfaction.

### BRIEF SUMMARY

Disclosed herein are personal care compositions with high naturality (e.g., personal care compositions with a naturality index greater than 95%) but which still retain the foaming performance of typically synthetic personal care formulations. Moreover, the personal care compositions of the disclosure can retain a certain level of thickness and structure without the inclusion of additional thickening agents. In one aspect, the personal care compositions of the disclosure are free of sulfate surfactants. In another aspect, in addition to being free of sulfates, the compositions have the benefit that they can be self-thickening. Consequently, in this aspect, the compositions of the disclosure are free of any thickener or polymer needed in the formulation. Without being bound by theory, certain combinations of an amphoteric surfactant (e.g., cocamidopropyl betaine) and long chain alkyl (C₆-C₂₂) anionic surfactants (e.g., disodium 2-sulfolaurate) in formulation can achieve desirable viscosity possibly by interactions of entangled worm-like micelles. In one aspect, and depending on the ingredients added and pH needed, the compositions comprise cocamidopropyl betaine (CAPB) and disodium 2-sulfolaurate (SFA) in a particular weight ratio between CAPB and SFA (e.g., between 0.85:0.15 to 0.3:0.7) to provide technical benefits and achieve desired performance.

In another aspect, all ingredients in the personal care compositions of the disclosure are natural or naturally derived. For example, in one aspect the ingredients can originate from coconut, palm, eucalyptus, lavender or bacterial fermentation. In yet another aspect, personal care compositions can be formulated without paraben, mineral oil, phthalate or optionally a preservative.

In one aspect, the disclosure provides a personal care composition (e.g., a liquid hand soap) comprising
An effective amount of a long chain alkyl (C₆-C₂₂) anionic surfactant (e.g., disodium 2-sulfolaurate);
An effective amount of an amphoteric surfactant (e.g., cocamidopropyl betaine),
An effective amount of one or more alkyl glucoside(s) (e.g., decyl glucoside and coco-glucoside); and
Water.

In another aspect, the invention provides a method of depositing a topically active compound on the skin, comprising applying an effective amount of any of personal care compositions as disclosed herein to the skin.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DESCRIPTION OF THE FIGURES

FIG. 1 describes the viscosity (cP) and micelle size (nm) of SFA/CAPB mixed micelle, at increasing molar fractions of CAPB in the mixture, at a pH of 4.5. Hydrodynamic diameter of SFA/CAPB mixed micelle is measured at a fixed total concentration of 35 mM. Viscosity is measured at a fixed total concentration of 140 mM.

FIG. 2 describes the viscosity (cP) and micelle size (nm) of SFA/CAPB mixed micelle, at increasing molar fractions of CAPB in the mixture, at a pH of 3.5. Hydrodynamic diameter of SFA/CAPB mixed micelle is measured at a fixed total concentration of 35 mM. Viscosity is measured at a fixed total concentration of 140 mM.

FIG. 3 describes the viscosity stability of the formulations listed in Table 1.

### DETAILED DESCRIPTION

The following description of various typical aspect(s) is merely exemplary in nature and is in no way intended to limit the disclosure, its application, or uses.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition. The amounts given are based on the active weight of the material.

The present invention provides, in an aspect, a personal care composition (Composition 1.0), (e.g., a liquid hand soap), comprising:
An effective amount of a long chain alkyl (C₆-C₂₂) anionic surfactant (e.g., disodium 2-sulfolaurate);
An effective amount of an amphoteric surfactant (e.g., cocamidopropyl betaine), and
An effective amount of one or more alkyl glucoside(s) (e.g., decyl glucoside) (e.g., decyl glucoside and coco-glucoside).

For example, the disclosure includes:
1.1. The personal care composition of Composition 1.0, wherein the long chain alkyl (C₆-C₂₂) anionic surfactant comprises a surfactant selected from the group consisting of: long chain alkyl sulfonates, long chain alkyl phosphates, long chain alkyl alpha olefin sulfonates, long chain alkyl taurates, long chain alkyl isethionates (SCl), long chain alkyl glyceryl ether sulfonates (AGES), sulfosuccinates, sodium methyl-2 sulfolaurate, disodium 2-sulfolaurate (e.g., TEXAPON® SFA produced by BASF), sodium lauryl sulfoacetate blend, long chain glutamates, long chain glycinates and combinations thereof.
1.2. The personal care composition of Composition 1.0 or 1.1, wherein the long chain alkyl (C₆-C₂₂) anionic surfactant comprises disodium 2-sulfolaurate (SFA) (e.g., from 0.5%-5% by wt.) (e.g., from 0.5%-2% by wt.) (e.g., about 0.75%) (e.g., about 1%) (e.g., about 2%) (e.g., about 2.5%).
1.3. The personal care composition of Composition 1.0 or 1.1, wherein the anionic surfactants are present in the composition in an amount of 0.5 to 15% by weight (e.g., about 0.75% by wt.) (e.g., about 1.3% by wt.).
1.4. Any of the foregoing personal care compositions, wherein the amphoteric surfactant is a sultaine amphoteric surfactant e.g., from 0.1%-15% by wt. of the total composition) (e.g., 0.5%-4% by wt. of the total composition) (e.g., about 3% by wt. of the total composition).
1.5. The preceding personal care composition, wherein the sultaine amphoteric surfactant (e.g., a hydroxysultaine) is selected from the group consisting of: lauramidopropyl hydroxysultaine, cocamidopropyl hydroxysultaine, oleamidopropyl hydroxysultaine, tallowamidopropyl hydroxysultaine, erucamidopropyl hydroxysultaine, lauryl hydroxysultaine, lauramidopropyl hydroxysultaine, and combinations thereof.
1.6. Any foregoing personal care compositions, wherein the amphoteric surfactant is a betaine amphoteric surfactant (e.g., from 0.1%-15% by wt. of the total composition) (e.g., 0.5%-4% by wt. of the total composition) (e.g., about 3% by wt. of the total composition) (e.g., about 4% by wt. of the total composition).
1.7. The preceding personal care composition, wherein the betaine amphoteric surfactant is a C8-C16 aminopropyl betaine (e.g., cocamidopropyl betaine)
1.8. The preceding personal care composition wherein the C8-C16 aminopropyl betaine is cocamidopropyl betaine.
1.9. The preceding personal care composition wherein the cocamidopropyl betaine, is present in an amount of from 0.5% to 20% by wt of the total composition.
1.10. The preceding personal care composition, wherein the cocamidopropyl betaine is from 1% to 5% by wt. of the total composition (e.g., about 4% by wt.).
1.11. Any of the preceding personal care compositions, wherein the alkyl glucoside is present in an amount of from 0.5% to 15%, e.g., from 1% to 8%, from 2% to 7%, by weight of the composition, optionally wherein the alkyl glucoside is present in an amount of from 1.5% to 8% by weight, from 1.5% to 7% by weight, or from 1.5% to 6.5% by weight of the composition.
1.12. Any of the preceding personal care compositions, wherein the alkyl glucoside is C₆₋₂₅ alkyl glucoside, e.g., C₈₋₁₈ alkyl glucoside, C₁₀₋₁₈ alkyl glucoside or C₁₀₋₁₆ alkyl glucoside, optionally wherein the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, arachidyl glucoside, and combinations thereof.
1.13. Any of the preceding personal care compositions, wherein the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, arachidyl glucoside, and combinations thereof (e.g., the alkyl glucoside is present in an amount of from 2% to 8% by weight, from 2% to 6% by weight, or from 2% to 4% by weight of the composition).
1.14. Any of the preceding personal care compositions, wherein the alkyl glucoside comprises decyl glucoside.
1.15. Any of the preceding personal care compositions, wherein the alkyl glucoside comprises coco-glucoside.
1.16. Any of the preceding personal care compositions, wherein the alkyl glucoside comprises a combination of decyl glucoside and coco-glucoside.
1.17. Any of the preceding personal care compositions, wherein the composition does not contain any surfactant other than the long chain alkyl (C₆-C₂₂) anionic surfactant, amphoteric surfactant and the alkyl glucoside.
1.18. Any of the preceding personal care compositions, wherein the total amount of surfactants present in the composition is from 5% to 30%, (e.g., from 5% to 15%), by weight of the composition.
1.19. Any of the preceding personal care compositions, wherein the composition comprises an oil selected from sunflower seed oil, olive oil, shear butter, jojoba oil, almond oil, grape seed oil, rose hip seed oil, mink oil, castor oil, soybean oil, mineral oil, and combinations thereof.
1.20. Any of the preceding personal care compositions, wherein the composition comprises a humectant,
1.21. The preceding composition wherein the humectant is selected from glycerin, sorbitol, and a combination thereof.
1.22. Any of the preceding personal care composition wherein composition comprises a carboxylic acid.
1.23. Any of the preceding personal care compositions wherein the carboxylic acid source comprises: citric acid, or lactic acid, or glycolic acid, or acetic acid, or succinic acid, or fumaric acid, or combinations thereof.
1.24. Any of the preceding personal care compositions wherein the carboxylic acid source comprises citric acid (e.g., from 0.05%-2% by wt. of the composition).
1.25. Any of the preceding personal care compositions, wherein the composition comprises water (e.g., from 10%-90% by wt.)(e.g., 25%-90%) (e.g., 50%-90%)(e.g., 50%-80%).
1.26. Any of the preceding composition, wherein the composition comprises:
   an effective amount of cocamidopropyl betaine (e.g., from 2.5%-20% by wt.) (e.g., about 4%);
   an effective amount of coco-glucoside (e.g., from 0.5%-10% by wt.) (e.g., about 1%) (e.g., about 2%);
   an effective amount of disodium 2-sulfolaurate (e.g., from 0.5%-5% by wt.) (e.g., about 0.75%) (e.g., about 1%) (e.g., about 2%) (e.g., about 2.5%); and
   water.
1.27. Any of the preceding composition, wherein the composition comprises:
   an effective amount of cocamidopropyl betaine (e.g., from 2.5%-20% by wt.) (e.g., about 4%);
   an effective amount of decyl glucoside (e.g., from 0.5%-5% by wt.) (e.g., about 1%) (e.g., about 2%);
   an effective amount of Disodium 2-sulfolaurate (e.g., from 0.5%-5% by wt.) (e.g., about 0.75%) (e.g., about 1%) (e.g., about 2%) (e.g., about 2.5%); and
water.
1.28. Any of the preceding composition, wherein the composition comprises:
   an effective amount of Cocamidopropyl betaine (e.g., from 2.5%-20% by wt.) (e.g., about 4%);
   an effective amount of Coco-glucoside (e.g., from 0.5%-10% by wt.) (e.g., about 1%) (e.g., about 2%);
   an effective amount of decyl glucoside (e.g., from 0.5%-5% by wt.) (e.g., about 1%) (e.g., about 2%);
   an effective amount of Disodium 2-sulfolaurate (e.g., from 0.5%-5% by wt.) (e.g., about 0.75%) (e.g., about 1%) (e.g., about 2%) (e.g., about 2.5%); and
   water.
1.29. Any of the preceding personal care compositions, wherein the composition is free or substantially free of sodium lauryl sulfate (SLS).
1.30. Any of the preceding personal care compositions, wherein the composition is free or substantially free of alkyl sulfate salts, e.g., C₁₋₂₅ alkyl sulfate salts.
1.31. Any of the preceding personal care compositions, wherein the composition is free or substantially free of sulfate.
1.32. Any of the preceding personal care compositions, wherein the composition further comprises an antiperspirant active, a deodorant active, an antioxidant, a fragrance, or a combination thereof.
1.33. Any of the preceding personal care compositions, wherein the composition is a rinse off composition, optionally wherein the composition is a liquid soap, liquid hand soap, shower gel, body wash, shampoo, or hair conditioner.
1.34. Any of the preceding personal care compositions, wherein the composition is a liquid hand soap.
1.35. Any of the preceding personal care compositions, wherein the composition is free or substantially free of any thickener.
1.36. Any of the preceding personal care compositions, wherein the composition is free or substantially free of paraben, mineral oil, phthalate and optionally a preservative.
1.37. Any of the preceding personal care compositions, wherein the composition comprises CAPB and SFA in a weight ratio (wt.% of the total composition) from 6:0.5 to 1:2 (CAPB:SFA) (e.g., 4.5:0.75 to 3.5:1.5) (e.g., about 4:1) (e.g., about 4:0.75).
1.38. Any of the preceding personal care compositions, wherein the composition comprises CAPB and SFA in a weight ratio (wt.% of the total composition) from 4.5:0.75 to 3.5:1.5 (e.g., about 4:1) (e.g., about 4:0.75) (CAPB: SFA).
1.39. Any of the preceding personal care compositions, wherein the ingredients are only derived from a coconut source, a palm source, a eucalyptus source, a lavender source, bacterial fermentation, and combinations thereof.
1.40. Any of the preceding personal care compositions, wherein the naturality index for the total composition is greater than 95% (e.g., about 98%).
1.41. Any of the preceding personal care compositions, wherein the pH of the composition is between 3.5-4.5 (e.g., from 3.8-4.2).
1.42. Any of the preceding personal care compositions, wherein the personal care composition comprises: about 4% cocamidopropyl betaine, from 2%-3.5% of one or more alkyl glucoside(s), wherein the alkyl glucoside comprises or consists of coco-glucoside, and from 0.5%-2.5% disodium 2-sulfolaurate (e.g., about 1.3%), wherein the weights are relative to the total composition.
1.43. Any of the preceding personal care compositions, wherein the personal care composition comprises: about 4% cocamidopropyl betaine, from 2%-3% of one or more alkyl glucoside(s), wherein the alkyl glucoside comprises or consists of decyl glucoside and coco-glucoside, and from 0.5%-2.5% disodium 2-sulfolaurate (e.g., about 0.75%), wherein the weights are relative to the total composition.

In one aspect, Composition 1 also includes the following:
1.44 A personal care composition comprising:
   a. An effective amount of a long chain alkyl (C₆-C₂₂) anionic surfactant;
   b. An effective amount of an amphoteric surfactant;
   c. An effective amount of one or more alkyl glucoside(s); and
   d. Water.
1.45 The personal care composition of 1.44, wherein the long chain alkyl (C₆-C₂₂) anionic surfactant comprises a surfactant selected from the group consisting of: long chain alkyl sulfonates, long chain alkyl phosphates, long chain alkyl alpha olefin sulfonates, long chain alkyl taurates, long chain alkyl isethionates (SCl), long chain alkyl glyceryl ether sulfonates (AGES), sulfosuccinates, sodium methyl-2 sulfolaurate, disodium 2-sulfolaurate (SFA) (e.g., TEXAPON® SFA produced by BASF), sodium lauryl sulfoacetate blend, and combinations thereof.
1.46 The personal care composition 1.44 or 1.45, wherein the long chain alkyl (C₆-C₂₂) anionic surfactant comprises disodium 2-sulfolaurate (SFA).
1.47 The personal care composition of any of 1.44-1.46, wherein the amphoteric surfactant is a betaine amphoteric surfactant.
1.48 The personal care composition of 1.47, wherein the C8-C16 aminopropyl betaine is cocamidopropyl betaine (CAPB).
1.49 The personal care composition of any of 1.44-1.48, wherein the alkyl glucoside is present in an amount of from 1% to 15%.
1.50 The personal care composition of 1.44-1.49, wherein the alkyl glucoside is C₆₋₂₅ alkyl glucoside.
1.51 The personal care composition of 1.44-1.50, wherein the alkyl glucoside comprises decyl glucoside.
1.52 The personal care composition of any of 1.44-1.51, wherein the alkyl glucoside comprises a combination of decyl glucoside and coco-glucoside.
1.53 The personal care composition of any of 1.44-1.52, wherein the composition comprises a carboxylic acid.
1.54 The personal care composition of any of 1.44-1.53, wherein the carboxylic acid source comprises: citric acid, or lactic acid, or glycolic acid, or acetic acid, or succinic acid, or fumaric acid, or combinations thereof.
1.55 The personal care composition of any of 1.44-1.54, wherein the composition comprises:
   a. an effective amount of cocamidopropyl betaine;
   b. an effective amount of coco-glucoside;
   c. an effective amount of disodium 2-sulfolaurate; and
   d. water.
1.56 The personal care composition of any of 1.44-1.55, wherein the composition comprises:
   a. an effective amount of cocamidopropyl betaine;
   b. an effective amount of decyl glucoside;
   c. an effective amount of disodium 2-sulfolaurate; and
   d. water.
1.57 The personal care composition of any of 1.44-1.56, wherein the composition comprises:
   a. an effective amount of cocamidopropyl betaine;
   b. an effective amount of coco-glucoside;
   c. an effective amount of decyl glucoside;
   d. an effective amount of disodium 2-sulfolaurate; and
   e. water.
1.58 The personal care composition of any of 1.44-1.57, wherein the composition is free or substantially free of sulfate.
1.59 The personal care composition of any of 1.44-1.58, wherein the composition is a rinse off composition, optionally wherein the composition is a liquid soap, liquid hand soap, shower gel, body wash, shampoo, or hair conditioner.
1.60 The personal care composition of 1.59, wherein the composition is a liquid hand soap.
1.61 The personal care composition of any of 1.44-1.60, wherein the composition is free or substantially free of any thickener.
1.62 The personal care composition of any of 1.44-1.61, wherein the composition is free or substantially free of paraben, mineral oil, phthalate and optionally a preservative.
1.63 Any of the preceding personal care compositions, wherein the composition comprises CAPB and SFA in a weight ratio (wt. % of the total composition) from 6:0.5 to 1:2 (CAPB: SFA) (e.g., 4.5:0.75 to 3.5:1.5) (e.g., about 4:1) (e.g., about 4:0.75).

The present invention provides, in another aspect, a personal care composition (Composition 2.0), e.g., any of Composition 1.0 et seq, for use in topical application to the skin.

Many personal care products incorporate oil such as sunflower seed oil, which provides more “crème like” appearance and/or other benefits. The addition of sunflower seed oil causes the initial formulation to go from a roughly homogenous mixture to an oil in water emulsion. In some embodiments, the composition comprises an oil selected from sunflower seed oil, olive oil, shear butter, jojoba oil, almond oil, grape seed oil, rose hip seed oil, mink oil, castor oil, soybean oil, mineral oil, and a combination thereof, optionally wherein the oil is sunflower seed oil.

In some embodiments, the composition (e.g., any of Composition 1.0 et seq or Composition 2.0 et seq) is free or substantially free of a thickener.

As used herein, “effective amount” refers to the amount of a compound or ingredient need to provide consumer acceptable thickening, foaming, and/or viscosity properties.

As used herein, “liquid hand soap” and “liquid hand wash” are used interchangeably.

In some embodiments, wherein the composition is free or substantially free of sodium lauryl sulfate (SLS). In some embodiments, the composition (e.g., any of Composition 1.0 et seq) is free or substantially free of sodium lauryl ether sulfate (SLES). In some embodiments, the composition is free or substantially free of sodium lauryl sulfate (SLS) and sodium lauryl ether sulfate (SLES). In some embodiments, the composition is free or substantially free of alkyl sulfate salts, e.g., C₁₋₂₅ alkyl sulfate salts. In some embodiments, the composition is free or substantially free of alkyl aryl sulfonate salts, e.g., C₁₋₂₅ alkyl aryl sulfonate salts, e.g., alkyl benzene sulfonate salts, e.g., sodium dodecyl benzene sulfonate. In some embodiments, the composition is free or substantially free of alkyl sulfate salts, e.g., C₁₋₂₅ alkyl sulfate salts, and alkyl aryl sulfonate salts, e.g., C₁₋₂₅ alkyl aryl sulfonate salts.

In some embodiments, e.g., any of Composition 1.0 et seq, the composition is free or substantially free of sulfate.

The composition of the present disclosure (e.g., any of Composition 1.0 et seq) may be any type of personal care composition. In certain embodiments, the composition is any composition that can be formulated into topical skin care formulations suitable for application to skin. Examples of such compositions include, but are not limited to, personal care compositions or skin care compositions, wherein “personal care composition” includes: antiperspirants, deodorants, liquid hand soap, body washes, creams, shower gels, bar soaps, shampoo, hair conditioners, and cosmetics. In some embodiments, the composition is a rinse off product (liquid soap, liquid hand soap, shower gel, body wash, shampoo, or hair conditioner, etc.). The composition (e.g., any of Composition 1.0 et seq) can comprise a single phase or can be a multi-phase system, for example a system comprising a polar phase and an oil phase, optionally in the form of a stable emulsion. The composition (e.g., any of Composition 1.0 et seq) can be liquid, semi-solid or solid. The formulation can be provided in any suitable container such as an aerosol can, tube or container with a porous cap, roll-on container, bottle, container with an open end, etc.

Water may be present in the compositions of the disclosure (e.g., any of Composition 1.0 et seq). Water employed in the preparation of commercial personal care compositions should be deionized and free of organic impurities. Water can make up the balance of the compositions and includes 10%-90%, or 10% to 80%, by weight of the personal care compositions. This amount of water includes the free water which is added plus that amount which is introduced with other materials such as glycerin, sorbitol or any components of the invention.

In some embodiments the composition of the disclosure (e.g., any of Composition 1.0 et seq) provides a sulfate-free personal care formulation comprising a surfactant system, humectants, antioxidants, pH adjusters, vitamins, fragrances, thickeners, oils, and water.

The pH adjusters of the present invention comprise but are not limited to lactic acid, sodium hydroxide, citric acid and combinations thereof.

The compositions of the present disclosure, e.g., any of Composition 1.0 et seq, provides a sulfate-free personal care formulation comprising an antibacterial agent. The antibacterial agent of the invention may be lactic acid.

The compositions of the present disclosure, e.g., any of Composition 1.0 et seq, contain a long chain alkyl anionic surfactant. In at least one aspect, the long chain alkyl anionic surfactant is a C₆-C₂₂ anionic surfactant that comprises a surfactant selected from the group consisting of: long chain alkyl sulfonates, long chain alkyl phosphates, long chain alkyl alpha olefin sulfonates, long chain alkyl taurates, long chain alkyl isethionates (SCl), long chain alkyl glyceryl ether sulfonates (AGES), sulfosuccinates, sodium methyl-2 sulfolaurate, disodium 2-sulfolaurate (e.g., TEXAPON® SFA produced by BASF), sodium lauryl sulfoacetate blend, and combinations thereof.

In some embodiments, Additional surfactants, which may be amphoteric or nonionic, and are known for use in personal care compositions, may be used as a co-surfactant. In some embodiments, the co-surfactant may comprise an alkyl glucoside. Alkyl glucoside is a compound produced by combining a sugar such as glucose with a fatty alcohol. Alkyl refers to unbranched or branched carbon chain. In some embodiments, the alkyl group is unbranched. The alkyl group may be saturated or unsaturated. In some embodiments, the alkyl group is saturated. For example, the structure of decyl glucoside is shown below:

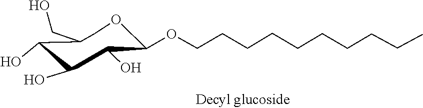

Alkyl glucoside may be C₈₋₂₅ alkyl glucoside, e.g., C₈₋₁₈ alkyl glucoside, C₁₀₋₁₈ alkyl glucoside or C₁₀₋₁₆ alkyl glucoside. In some embodiments, the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, arachidyl glucoside, and a combination thereof. In some embodiments, the alkyl glucoside is selected from decyl glucoside, caprylyl/capryl glucoside and a combination thereof. In some embodiments, the weight ratio of caprylyl/capryl glucoside to decyl glucoside present in the composition is 2 to 1 or 4 to 1, e.g., about 3 to 1. In some embodiments, the alkyl glucoside is present in an amount of from 1% to 9%, e.g., from 2% to 8%, from 2% to 7%, by weight of the composition.

In some embodiments, the compositions of the disclosure (e.g., any of Composition 1.0 et seq) can comprise a C₈-C₁₆ aminopropyl betaine surfactant, e.g., cocamidopropyl betaine. In some embodiments, the compositions of the disclosure (e.g., any of Composition 1.0 et seq) can comprise a non-ionic block copolymer, optionally together with an alkyl glucoside. The non-ionic block copolymer may be a poly(propylene oxide)/poly(ethylene oxide) copolymer. In some embodiments, the copolymer has a polyoxypropylene molecular mass of from 3000 to 5000 g/mol and a polyoxyethylene content of from 60 to 80 mol %. In some embodiments, the non-ionic block copolymer is a poloxamer. In some embodiments, the non-ionic block copolymer is selected from: Poloxamer 338, Poloxamer 407, Poloxamer, 237, Poloxamer, 217, Poloxamer 124, Poloxamer 184, Poloxamer 185, and a combination of two or more thereof. In some embodiments, the copolymer is Poloxamer 407. In some embodiments, the compositions of the disclosure (e.g., any of Composition 1.0 et seq) can comprise a betaine amphoteric surfactant and a non-ionic block copolymer, optionally together with an alkyl glucoside.

In some embodiments, the personal care composition of the disclosure (e.g., any of Composition 1.0 et seq) may be free or substantially free of sodium lauryl sulfate (SLS) and/or sodium lauryl ether sulfate (SLES). In some embodiments, the composition may be free or substantially free of alkyl sulfate salts. In some embodiment, the alkyl sulfate salts are C₁₋₂₅ alkyl sulfate which may be saturated or unsaturated, and unbranched or branched. In some embodiments, the personal care composition may be free or substantially free of alkyl aryl sulfonate salts, e.g., alkyl benzene sulfonate salts, e.g., sodium dodecyl benzene sulfonate. In some embodiments, the personal care composition may be free or substantially free of alkyl sulfate salts and alkyl aryl sulfonate salts. In some embodiments, the composition may be free or substantially free of sulfate. As used herein, “substantially free” of a material may refer to a composition where the material is present in an amount of less than 0.1 weight %, less than 0.05 weight %, less than 0.01 weight %, less than 0.005 weight %, less than 0.001 weight %, or less than 0.0001 weight % based on a total weight of the composition.

In some aspects, the personal care compositions of the present disclosure, (e.g., any of Composition 1.0 et seq), comprise one or more topically active compounds. Topically active compounds may encompass a wide range of materials, including antibacterial agents, vitamins, medicaments, fragrance materials, antioxidants, antiperspirant actives, deodorant actives, and other skin-care ingredients. In some aspects the personal care composition of the present disclosure, e.g., any of Composition 1.0 et seq, comprises a vitamin. Illustrative vitamins may be or include, but are not limited to, vitamin C, vitamin D, vitamin E, vitamin K, and a combination thereof.

In certain aspects, optional ingredients of the personal care compositions of the disclosure, e.g., any of Composition 1.0 et seq, include solvents; water-soluble alcohols such as C₂₋₈ alcohols including ethanol; glycols including propylene glycol, dipropylene glycol, tripropylene glycol and mixtures thereof; glycerides including mono-, di- and triglycerides; medium to long chain organic acids, alcohols and esters; surfactants including emulsifying and dispersing agents; amino acids including glycine; emollients; fragrances; and colorants including dyes and pigments.

In certain aspects, the compositions of the disclosure, e.g., any of Composition 1.0 et seq, may optionally contain emollients in any desired amount to achieve a desired emollient effect. Emollients are known in the art and are used to impart a soothing effect on the skin. Non-volatile emollients are preferable. Classes of non-volatile emollients include non-silicone and silicone emollients. Non-volatile, non-silicone emollients include C₁₂₋₁₅ alkyl benzoate. The non-volatile silicone material can be a polyethersiloxane, polyalkyarylsiloxane or polyethersiloxane copolymer. An illustrative non-volatile silicone material is phenyl trimethicone. Examples include, but are not limited to, PPG-14 butyl ether, PPG-3 myristyl ether, secondary alcohol ethoxylates, stearyl alcohol, stearic acid and salts thereof, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, sunflower seed oil, acetylated lanolin alcohols, acetylated lanolin, petrolatum, isopropyl ester of lanolin, fatty acids, mineral oils, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide. In some embodiments, the composition of the disclosure, e.g., any of Composition 1.0 et seq, comprises an oil selected from sunflower seed oil, olive oil, shear butter, jojoba oil, almond oil, grape seed oil, rose hip seed oil, mink oil, castor oil, soybean oil, mineral oil, and a combination thereof. In certain embodiment, the composition comprises sunflower seed oil.

In certain aspects, the composition (e.g., any of Composition 1.0 et seq) may include one or more humectants. Humectants can reduce evaporation and also contribute towards preservation by lowering water activity and can also impart desirable sweetness or flavor to compositions. Illustrative humectants may be or include, but are not limited to, glycerin, propylene glycol, polyethylene glycol, sorbitol, xylitol, or the like, or any mixture or combination thereof. In some embodiment, the humectant is selected from glycerin, sorbitol and a combination thereof. In certain embodiment, the humectant is glycerin.

Antioxidants may be added to the composition, preferably to act as ingredient protectants and for maintenance of long-term stability of the composition. Examples of antioxidants include, but are not limited to citric acid, butylated hydroxytoluene, pentaerythrityl tetra-di-t-butyl hydroxyhydrocinnamate.

In certain aspects, the compositions of the disclosure (e.g., any of Composition 1.0 et seq) may optionally contain polymeric materials for thickening, such as natural or synthetic gums, such as polyglycerides including agar, agarose, pectin, or guars, polysaccharides, associative thickeners (e.g., PEG150 distearate) or combinations thereof. One class of materials worthy of attention for thickening a water-immiscible phase comprises derivatives of hydrolysed starch or other polysaccharides, including in particular esterified dextrins, such as dextrin palmitate. Suspending agents such as silicas or clays such as bentonite, montmorillonite or hectorite, including those available under the trademark Bentone can also be employed to thicken liquid compositions according to the invention. The composition can be thickened with non-polymeric organic gellants, including selected dibenzylidene alditols (e.g., dibenzylidene sorbitol).

Fragrance may be included in the personal care composition of the disclosure, e.g., any of Composition 1.0 et seq. Any fragrance suitable for personal care use may be incorporated into the personal care composition of the invention. Fragrances tend to be relatively volatile aroma compounds which are capable of entering the gas phase at skin surface temperature.

The personal care compositions of the invention may be manufactured using methods known in the art. Typically, the ingredients are combined and optionally heated where components need to be melted. The components are mixed. Desirably, volatile materials such as fragrant materials are incorporated in the composition in the latter stages of a mixing cycle in order to avoid volatilization thereof. After mixing, the composition may be poured directly into the dispensers and the container capped to preserve the product until use.

In another aspect, the invention provides a method of depositing a topically active compound on the skin, comprising applying an effective amount of any of personal care compositions disclosed herein, e.g., any of Compositions 1 et seq., to the skin.

### EXAMPLES

### Example 1

Evaluation of Optimal Surfactant Ratio

In a polymer-free and thickener-free surfactant formulation a key parameter to increasing solution viscosity is the entanglement of wormlike micelles. Without being bound by theory, the greater the micelles swell in diluted solution, the more likely the worm-like micelles will elongate and entangle with each other thus to build viscosity at elevated concentration.

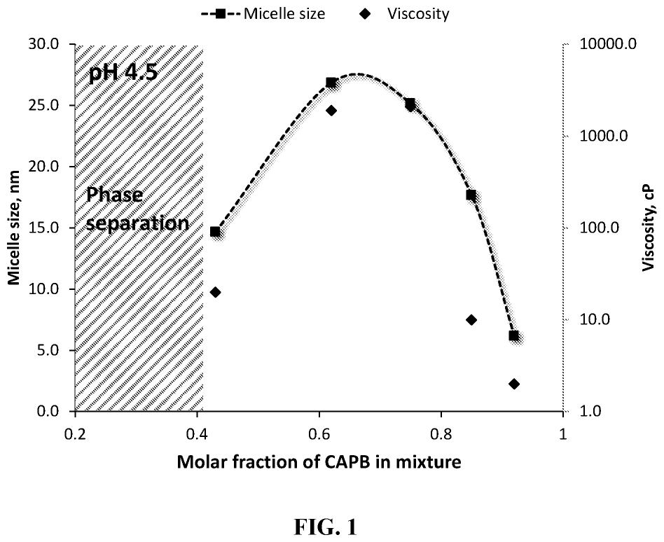

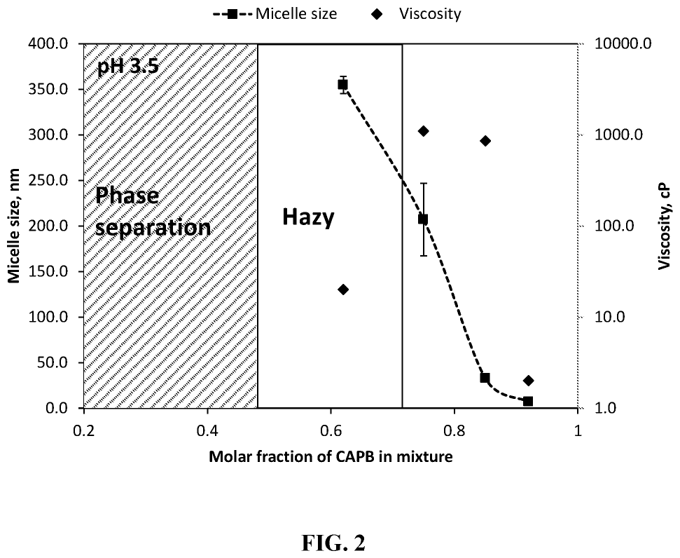

The optimal ratio between SFA/CAPB is screened via measuring micelle size by dynamic light scattering while maintaining total surfactants concentration at about 35 mM (˜1.2% Active Ingredient “AI”). At such low concentration, all studied solutions are water-like, i.e., having very low viscosity. As seen in FIG. 1, at a pH of 4.5, the SFA/CAPB mixed micelle have the largest hydrodynamic diameter at CAPB ratio around 0.7±0.1, corresponding to the maximum viscosity attained at elevated surfactant concentration of 140 mM (˜5.0% AI). At a pH of 3.5, and without being bound by theory, it is believed that because of the protonation of the carboxylate group, CAPB possesses net positive charge and strongly interacts with anionic SFA. The resulting mixture micelle swells most and easily forms large irregular structure or even phase separation at CAPB ratio≤0.7. Combined with the measured viscosity, FIG. 2 demonstrate the optimal ratio of CAPB was determined around 0.85 at pH of 3.5.

As observed in FIG. 1 and FIG. 2, optimal CAPB ratio in CAPB/SFA mixture increases from 0.7±0.1 to 0.85 when pH decreases from 4.5 to 3.5. At pH of 3.8-4.2, the optimal ratio of CAPB in SFA/CAPB mixture is easily determined ˜0.8.

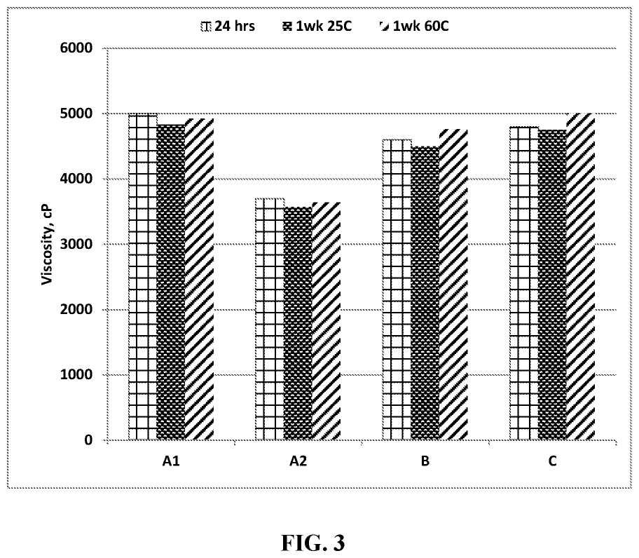

Table 1 provides test formulations with high natural origin index (“NOI”) (NOI≥0.980) that are designed to verify the optimal ratio. All formulated batch samples have viscosity between 3500-5000 cP. Considering the relatively low AI (4.97-6.00%) of surfactants in these formulations, the viscosities achieved are acceptable. As shown in FIG. 3, viscosities of all formulations are stable over 1-week aging at both 25° C. and 60° C.

TABLE 1a
Example formulation containing SFA/CAPB at pH ~4.0-4.2

| | | | | |
|---|---|---|---|---|
| Ingredient | Wt. % | AI | NOI | % NOI |

Test Formulation A1: SFA/CAPB high AI

| | | | | |
|---|---|---|---|---|
| DI water | 77.90 | — | 1.00 | 0.78 |
| CAPB | 15.00 | 4.50 | 0.89 | 0.13 |
| SFA | 3.90 | 1.17 | 1.00 | 0.04 |
| Glycerin | 2.00 | — | 0.98 | 0.02 |
| Fragrance | 0.20 | — | 1.00 | 0.00 |
| Citric acid | 1.00 | — | 1.00 | 0.01 |
| Total | 100.00 | 5.67 | — | 0.983 |
| pH | 4.2 | — | — | — |

Test Formulation A2: SFA/CAPB low AI

| | | | | |
|---|---|---|---|---|
| DI water | 80.22 | — | 1.00 | 0.80 |
| CAPB | 13.20 | 3.96 | 0.89 | 0.12 |
| SFA | 3.38 | 1.01 | 1.00 | 0.03 |
| Glycerin | 2.00 | — | 0.98 | 0.02 |
| Fragrance | 0.20 | — | 1.00 | 0.00 |
| Citric acid | 1.00 | — | 1.00 | 0.01 |
| Total | 100.00 | 4.97 | — | 0.985 |
| pH | 4.20 | — | — | — |

Test Formulation B: CAPB/SFA/Sultaine

| | | | | |
|---|---|---|---|---|
| DI water | 78.82 | — | 1.00 | 0.79 |
| Sultaine | 1.32 | 0.50 | 0.76 | 0.01 |
| CAPB | 13.33 | 4.00 | 0.89 | 0.12 |
| SFA | 3.33 | 1.00 | 1.00 | 0.03 |
| Glycerin | 2.00 | — | 0.98 | 0.02 |
| Fragrance | 0.20 | — | 1.00 | 0.00 |
| Citric acid | 1.00 | — | 1.00 | 0.01 |
| Total | 100.00 | 5.50 | — | 0.982 |
| pH | 4.0 | — | — | — |

Test Formulation C: CAPB/SFA/Decyl Glucoside

| | | | | |
|---|---|---|---|---|
| DI water | 78.13 | — | 1 | 0.78 |
| CAPB | 13.33 | 4.00 | 0.89 | 0.12 |
| SFA | 3.33 | 1.00 | 1 | 0.03 |
| Decyl Glucoside | 2.00 | 1.00 | 1 | 0.02 |
| Glycerin | 2.00 | — | 0.98 | 0.02 |
| Fragrance | 0.20 | — | 1 | 0.00 |
| Citric acid | 1.00 | — | 1 | 0.01 |
| Total | 100.00 | 6.00 | — | 0.985 |
| pH | 4.2 | — | — | — |

Table 2—Foamability

The foamability of Formulation C is measured by Kruss DFA and compared against a commercially available product, which serves as a control, and contains both SLES and CAPB. The composition details of the control is shown is Table 1b. The key parameters of foamability are summarized in Table 2. The foam generation and foam stability of Formulation A1, A2, and B are comparable to the control, while Formulation C is superior than control.

TABLE 1b
Control Formulation of Shower Gel Product

| Ingredient | Wt. % |
|---|---|
| Water | q.s. (e.g., 86.4) |
| Sodium laureth sulfate | 8.5 |
| Cocamidopropyl betaine | 2.5 |
| Cocamide MEA | 0.5 |
| Sodium chloride | 0.5 |
| Citric acid | 0.2 |
| Preservative | 0.3 |
| Remainder | 1.1 |
| Total | 100.0 |

TABLE 2
Comparison of foam parameters

| | tₘₐₓ, s | V_{foam max}, mL | q_{foam}, mL/s |
|---|---|---|---|
| Formulation A1 | 31.01 | 49.90 | 1.61 |
| Formulation A2 | 31.01 | 53.60 | 1.73 |
| Formulation B | 31.01 | 41.30 | 1.33 |
| Formulation C | 31.01 | 62.00 | 2.00 |
| (Control) | 31.01 | 47.20 | 1.52 |

* tₘₐₓ, time of maximum foam height in seconds; V_{foam max}, maximum foam, volume; q_{foam}, flash foam rate.

### Example 2

### Comparison of Test Formulations

TABLE 2
Formulations 1 to 5

| Ingredient (wt %) | 1 | 2 | 3 | 4 | 5 |
|---|---|---|---|---|---|
| Cocamidopropyl | 10 | 12 | 12 | 13.5 | 13 |
| Betaine (30 wt %) | | | | | |
| Decyl glucoside | 4 | 5.6 | 5.6 | — | 2 |
| (50 wt %) | | | | | |
| Coco-glucoside | — | — | — | 4.5 | 4 |
| (50 wt %) | | | | | |
| Glycerin | 2 | 2 | 2 | 2 | 2 |
| SFA - Powder | 4 | 2.1 | 2.1 | — | 2.1 |
| SFA - Paste | — | — | — | 2.5 | — |
| Xanthan Gum | — | — | — | — | 0.02 |
| Lactic Acid | 0.4 | 0.8 | — | — | — |
| Citric Acid | — | — | 0.7 | 1 | 1 |
| Water | q.s. | q.s. | q.s. | q.s. | q.s. |
| Sodium Chloride | 1.4 | 0.1 | — | — | — |
| Fragrance | 0.2 | 0.2 | 0.2 | 0.2 | 0.2 |
| Total | 100% | 100% | 100% | 100% | 100% |
| Viscosity in | >2200 | >1300 | >1320 | >2400 | >3350 |
| Accelerated Aging/cps | | | | | |
| Foam Volume in | 74 | 72 | 73 | 74 | 73 |
| Foam spin/ml | | | | | |

Formula 4 above demonstrates similar foam volume—i.e., parity foaming performance—when compared to a commercially available natural liquid hand wash (foam volume 76 ml, viscosity 2160 cps). After eight weeks of an accelerated stability test, the Formula 4 demonstrates acceptable stability. The commercially available natural liquid hand wash is believed to contain cocamidopropyl betaine and decyl glucoside, but not SFA.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1. A personal care composition comprising:
a. An effective amount of a long chain alkyl (C₆-C₂₂) anionic surfactant;
b. An effective amount of an amphoteric surfactant;
c. An effective amount of one or more alkyl glucoside(s); and
d. Water.

2. The personal care composition of claim 1, wherein the long chain alkyl (C₆-C₂₂) anionic surfactant comprises a surfactant selected from the group consisting of: long chain alkyl sulfonates, long chain alkyl phosphates, long chain alkyl alpha olefin sulfonates, long chain alkyl taurates, long chain alkyl isethionates (SC1), long chain alkyl glyceryl ether sulfonates (AGES), sulfosuccinates, sodium methyl-2 sulfolaurate, disodium 2-sulfolaurate (SFA), sodium lauryl sulfoacetate blend, and combinations thereof.

3. The personal care composition of claim 1, wherein the long chain alkyl (C₆-C₂₂) anionic surfactant comprises disodium 2-sulfolaurate (SFA).

4. (canceled)

5. The personal care composition of claim 1, wherein the amphoteric surfactant is a betaine amphoteric surfactant.

6. The personal care composition of claim 5, wherein the C8-C16 aminopropyl betaine is cocamidopropyl betaine (CAPB).

7. The personal care composition of claim 1, wherein the alkyl glucoside is present in an amount of from 1% to 15%.

8. The personal care composition of claim 1, wherein the alkyl glucoside is C₆₋₂₅ alkyl glucoside.

9. (canceled)

10. The personal care composition of claim 1, wherein the alkyl glucoside comprises decyl glucoside.

11. The personal care composition of claim 1, wherein the alkyl glucoside comprises a combination of decyl glucoside and coco-glucoside.

12. (canceled)

13. The personal care composition of claim 1, wherein the composition comprises a carboxylic acid.

14. The personal care composition of claim 1, wherein the carboxylic acid source comprises: citric acid, or lactic acid, or glycolic acid, or acetic acid, or succinic acid, or fumaric acid, or combinations thereof.

15. The personal care composition of claim 1, wherein the composition comprises:
a. an effective amount of cocamidopropyl betaine;
b. an effective amount of coco-glucoside;
c. an effective amount of disodium 2-sulfolaurate; and
d. water.

16. The personal care composition of claim 1, wherein the composition comprises:
a. an effective amount of cocamidopropyl betaine;
b. an effective amount of decyl glucoside;
c. an effective amount of disodium 2-sulfolaurate; and
d. water.

17. The personal care composition of claim 1, wherein the composition comprises:
a. an effective amount of cocamidopropyl betaine;
b. an effective amount of coco-glucoside;
c. an effective amount of decyl glucoside;
d. an effective amount of disodium 2-sulfolaurate; and
e. water.

18. The personal care composition of claim 1, wherein the composition is free or substantially free of sulfate.

19. The personal care composition of claim 1, wherein the composition is a rinse off composition, optionally wherein the composition is a liquid soap, liquid hand soap, shower gel, body wash, shampoo, or hair conditioner.

20. The personal care composition of claim 19, wherein the composition is a liquid hand soap.

21. The personal care composition of claim 1, wherein the composition is free or substantially free of any thickener.

22. The personal care composition of claim 1, wherein the composition is free or substantially free of paraben, mineral oil, phthalate and optionally a preservative.

23. The personal care composition of claim 1, wherein the composition CAPB and SFA in a weight ratio (wt.% of the total composition) from 6:0.5 to 1:2 (CAPB:SFA).
